(19) United States
(12) Patent Application Publication
(10) Pub. No.: US 2008/0140091 A1
(43) Pub. Date: Jun. 12, 2008

(54) MINIMALLY INVASIVE SUTURE-BASED REPAIR OF SOFT TISSUE

(76) Inventors: Patrick G. DeDeyne, Duxbury, MA (US); Mark Hall, Bridgewater, MA (US); Michael J. O'Neil, West Barnstable, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/609,383

(22) Filed: Dec. 12, 2006

(51) Int. Cl. A61B 17/062

(52) U.S. Cl. 606/144

(57) ABSTRACT

This invention is directed to an instrument that passes a suture through the annulus fibrosus or other soft tissue and that can be used in a minimally invasive manner.

### BACKGROUND OF THE INVENTION

1. Field of the Invention

This invention is concerned with repair of soft tissue, particularly with repair of the annulus fibrosus of an intervertebral disc or meniscus with a novel suturing device and method.

2. Related Art

Back pain is a major cause for loss of work and represents a significant portion of health care expenditures. The prevalence of low back pain is reported to range from 7% to 37% depending on the population. While several causes are thought to be associated with back pain such as genetic factors and psychological factors a relative large cohort of patients with back pain have anatomic pathologies in the intervertebral disc (IVD) leading to herniation of IVD material thereby compressing a nerve root, which requires surgical decompression (discectomy). Furthermore, about 8-30% of the patients re-herniate at the same spinal level, requiring revision surgery.

The current surgical practice entails removal of the bulging IVD material without giving any consideration to the remaining IVD tissue. However, it is very likely that future procedures may include repair of the intervertebral disc including suturing of the outer annulus fibrosus. The main obstacle for this approach is the lack of instrumentation that would allow suturing the annulus fibrosis during a minimally invasive approach. Extensive experience in arthroscopic surgery of the knee and especially shoulder joint have led to instruments that allow the passing of sutures in an area that is just a few cm³ large. To date, no such instrumentation has been described for the use of suture-based repair of the annulus fibrosus.

Several companies that focus on instrumentation for arthroscopic evaluation and surgery for the shoulder joint have suture passers. The Arthrex Co. has two instruments Viper™ and Scorpion™ and DePuy MITEK has Expressew™. Correspondingly, several patents related to these instruments do disclose the use of suture passing instruments including U.S. Pat. No. 6,984,237, U.S. Pat. No. 6,626,929 U.S. 2003/0065337, U.S. 2004/0199184, 2003/0083695, U.S. Pat. No. 5,947,982, U.S. Pat. No. 6,051,006, and U.S. 2005/0288690 however they do not pertain to the surgical repair of the intervertebral disc.

The use of specific instrumentation for suture-based repair of the intervertebral disc and more specifically the annulus fibrosus has not been disclosed. However, the idea of suture-based repair is not new and actually Cauthen (U.S. 2003/0158604 A1) teaches extensively on different approaches on suture-based (with or without anchors) repair of the annulus fibrosus. An instrument that inserts a suture into the intervertebral disc by shielding the suture, then clamping the annulus through a minimally invasive approach to subsequently pass the suture through the annulus has not been disclosed.

Cauthen (U.S. 2003018604 and U.S. 20050283246) discloses using sutures and suture anchors without clamping annular opening together while passing the suture through the lateral annulus. Yeung (U.S. Pat. No. 6,530,933) discloses the use of sutures to compress the a IVD herniation by placing a suture anchor into opposite annular wall and extending sutures across the disc space to secure. Keith (U.S. 20050049592) discloses use of sutures with a reinforcement member, but does not disclose clamping annular opening together while passing the suture through the annulus.

A few patents mention the use of sutures to attach a degradable or a non-degradable device into or onto the intervertebral disc (e.g. Ferree in US2005/0124992 A1; Malaviya in US2004/014334A1; Zucherman US2005/0209603 A1; Cauthen US2003/0220690 A1). However, none of the patents that are published describes a suturing device that has independently pivoting jaws or teaches a method how to insert a suture into a disc using a minimally invasive approach clamp the annulus together while passing the suture through the annulus for subsequent closure. We disclose this minimally invasive instrument that can be used for suture-based repair of soft tissue, particularly the meniscus of the knee and the intervertebral disc and the annulus fibrosus in particular to assist in repair of the annulus as well as containment of nuclear treatments.

### BRIEF DESCRIPTION OF THE DRAWINGS

FIG. 1 depicts an embodiment of this invention wherein the tissue grasping jaws are shown in a closed position and the flexible needle is in the retracted position.

FIG. 2 depicts an embodiment of this invention wherein the tissue grasping jaws are shown in an open position and the flexible needle is in the retracted position.

FIG. 3 depicts an embodiment of this invention wherein the tissue grasping jaws are shown in a closed position and the flexible needle is in the retracted position.

FIG. 4 depicts an embodiment of this invention wherein the tissue grasping jaws are shown in a closed position and the flexible needle is in the extended position.

FIGS. 5 a-f depict various steps in the use of this invention.

FIGS. 6 a-b show various flexible needle tip configurations for acceptance of suturing material.

### SUMMARY OF THE INVENTION

This invention is directed to a minimally invasive device for suture repair of soft tissue comprising:

a) an elongated housing having a proximal end and a distal end;

b) the distal end comprising a suturing assembly comprising:
(i) an upper jaw and a lower jaw wherein the upper and lower jaws are pivotally mounted with respect to each other and with respect to the housing;
(ii) a flexible needle adapted to carry a suture, the needle being movable between a first position and a second position and wherein the needle is substantially housed within and extendable from and retractable within one of the jaws and the housing and extends to the proximal end of the housing through the housing; and

c) the proximal end comprising a handle, a needle actuator and a jaws actuator, the needle actuator connected to the needle for extension and retraction of the needle from the needle-containing jaw, and the jaws actuator linked to the jaws within the housing for closing and opening of the jaws with respect to each other and for grasping and releasing the tissue to be sutured.

Further embodiments of the invention relate to a method of suturing soft tissue comprising the steps of:

i) providing tissue to be sutured;

ii) passing a needle carrying a suture through the tissue using a device having two independently pivotable tissue grasping jaws, wherein the first jaw houses the needle and provides a channel for the needle to have a retracted storage position and an extended tissue piercing position and the second jaw contains a channel for receiving the needle and suture after the needle and suture has passed through the tissue; and

iii) grasping and tying the suture after the suture has been passed through the tissue.

### DETAILED DESCRIPTION OF PREFERRED EMBODIMENTS OF THE INVENTION

The present invention is directed toward minimally invasive suturing of tissue, particularly soft tissue. As used herein, the term soft tissue is intended to describe tissue that is penetrable by a needle and otherwise capable of being sutured. Soft tissues are tissues that surround, connect, and support organs. Typical examples are muscle, tendon, ligament, fascia, and aponeurosis. However, most organs for example the heart, lungs, brain, internal organs, viscera, blood vessels, and nerves contain soft tissue around the organ (i.e., pericardium, perineurium, or dura mater). During surgical repair of these organs soft tissues the soft tissue envelope is breached and needs to be re-approximated or re-attached, in which suturing may be the method of choice. Tissues such as the menisci, pubic disc, or intervertebral disc are typical examples of fibro-cartilage, which can also be sutured to re-approximate or repair severed end of the tissue. The most preferred tissues that this invention is directed to is the suturing of curvilinearly shaped tissue such as an intervertebral disc and menisci.

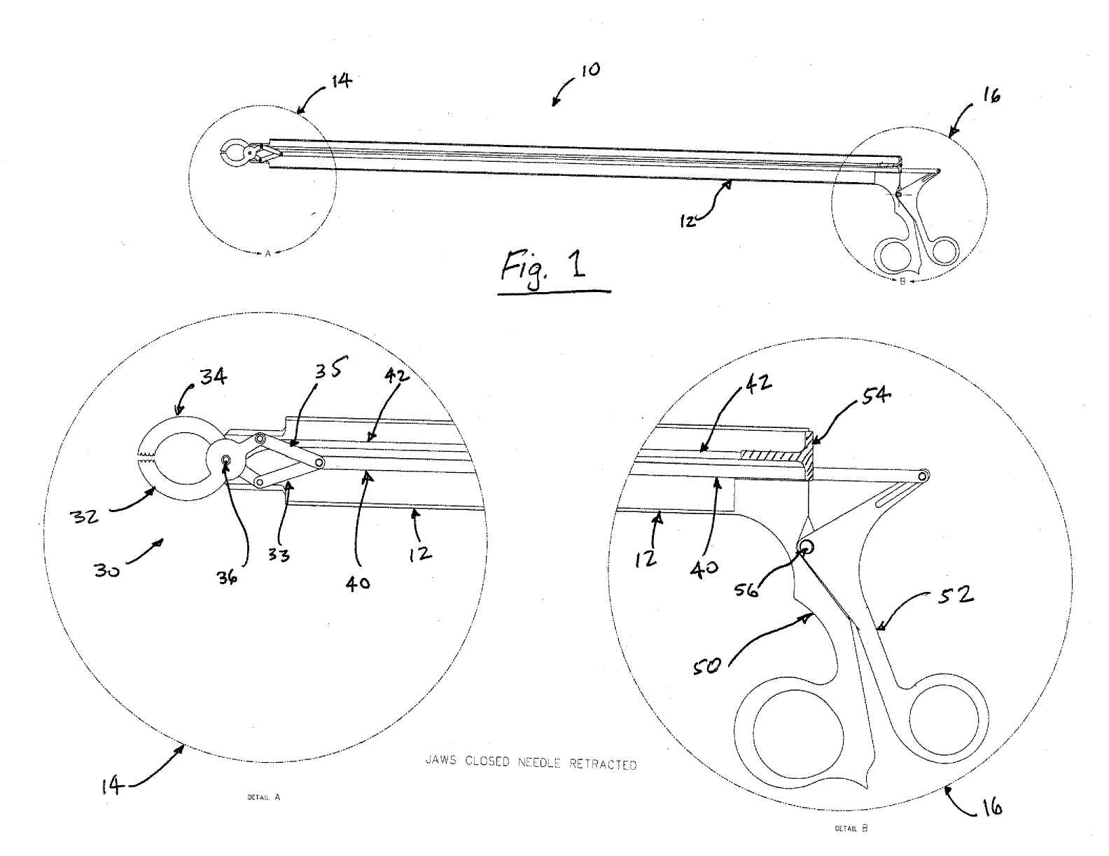

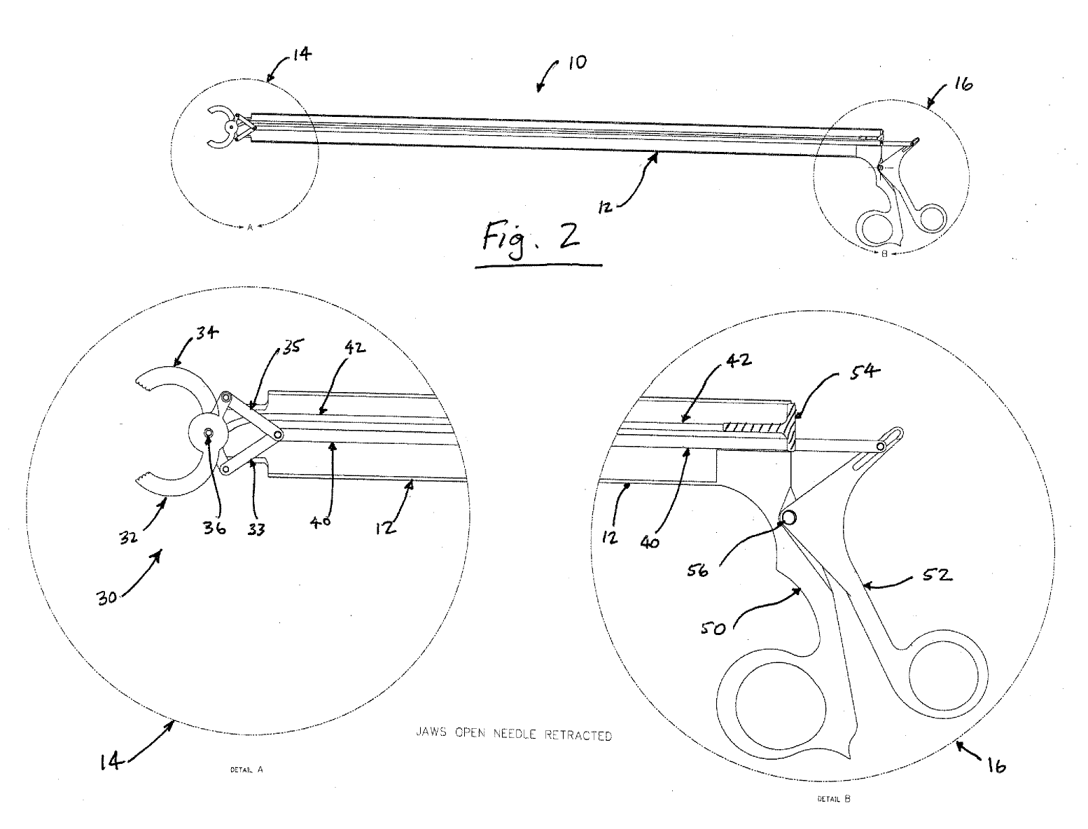

The minimally invasive aspect of this invention refers to the fact that the device sutures tissue in a fashion that minimizes disturbance or damage to other tissue by virtue of the fact the device of this invention can be deployed through a cannula. The term minimally invasive surgery (MIS) is intended to include procedures that utilize small incisions through which cameras and instruments are inserted and accomplish the operation. In MIS the operation may be followed by video or even can be performed via a robotic arm. In most, if not all, cases, small instruments are required, which allow the surgeon to perform certain tasks from a distance away from the wound or surgical repair site (e.g., closing tissue with sutures). The advantages of MIS are several: smaller incisions, less scarring, shorter hospital stay, shorter rehabilitation, and faster return to full activities of daily living. On the other hand it requires more extensive and unique training for surgeons and operating room staff. Thus, reference to FIG. 1 illustrates the minimally invasive nature of the device of this invention as it can be seen that the grasping jaws of the suturing assembly part of the device are within the profile of the elongated shaft of the device, therefore making the device easily insertable through a cannula to the desired tissue suturing site.

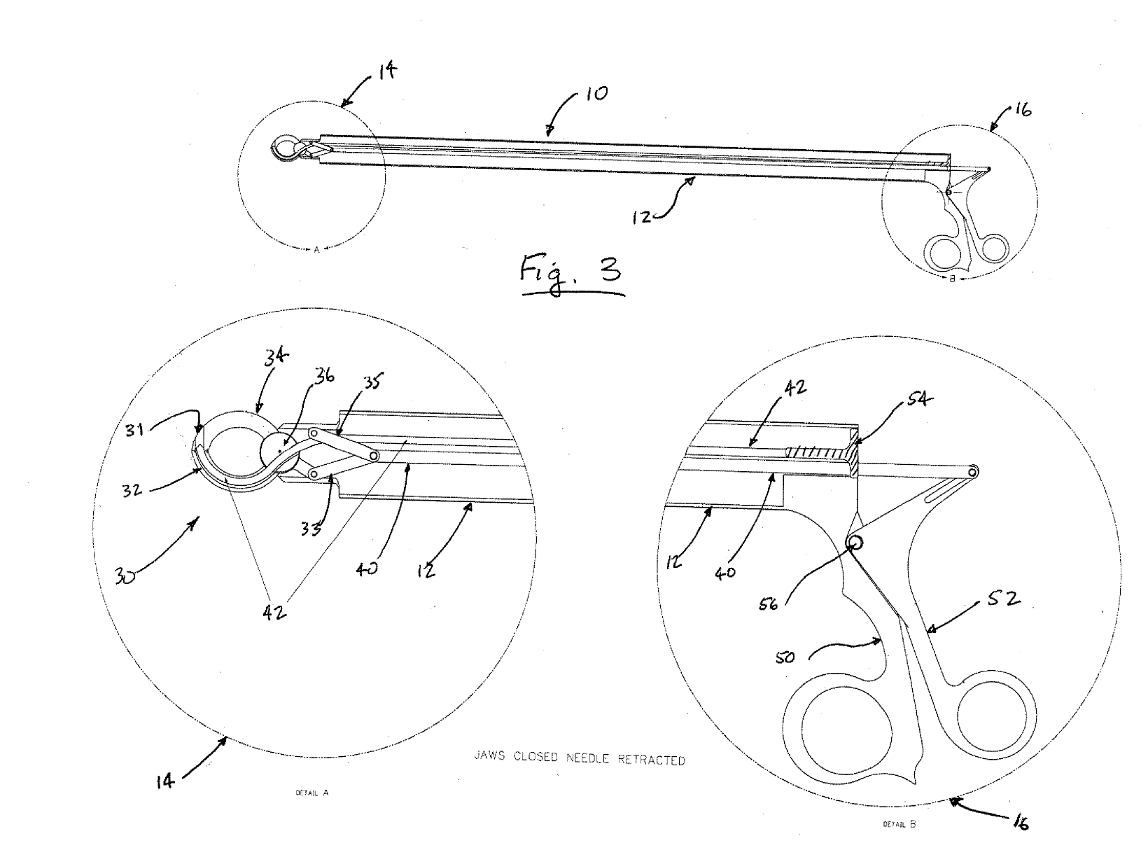

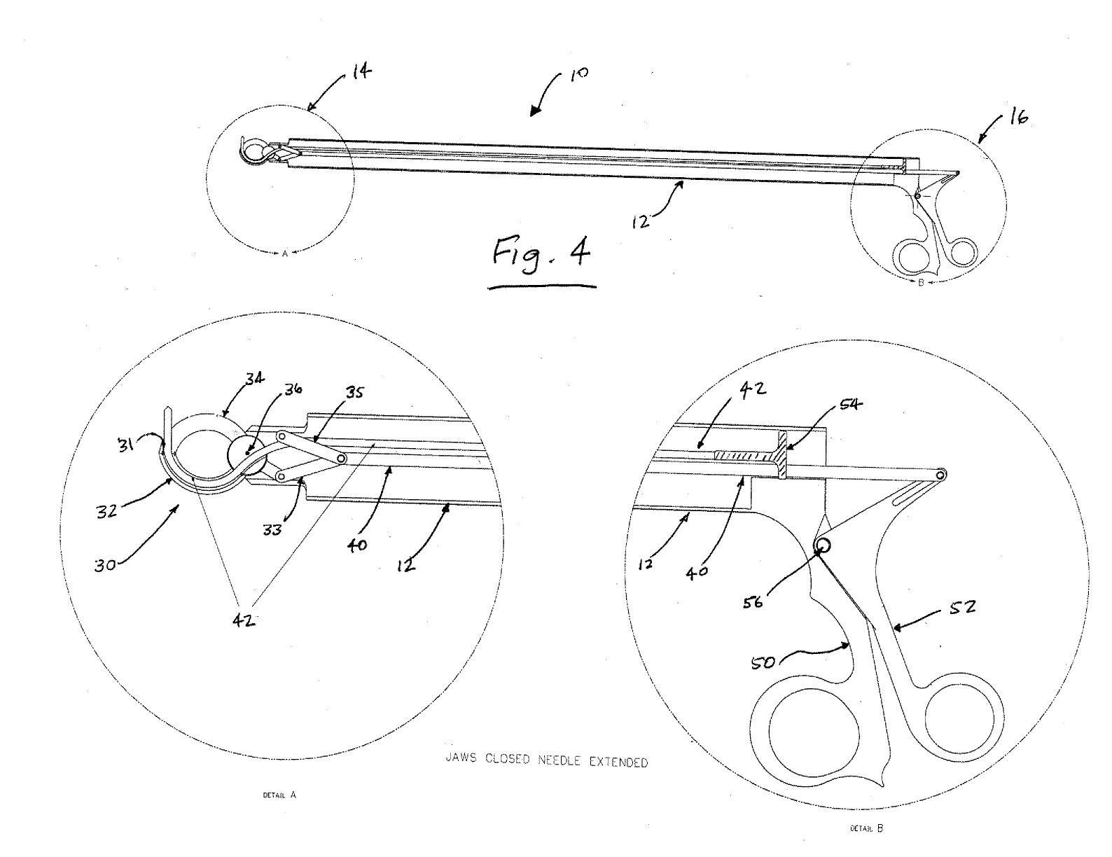

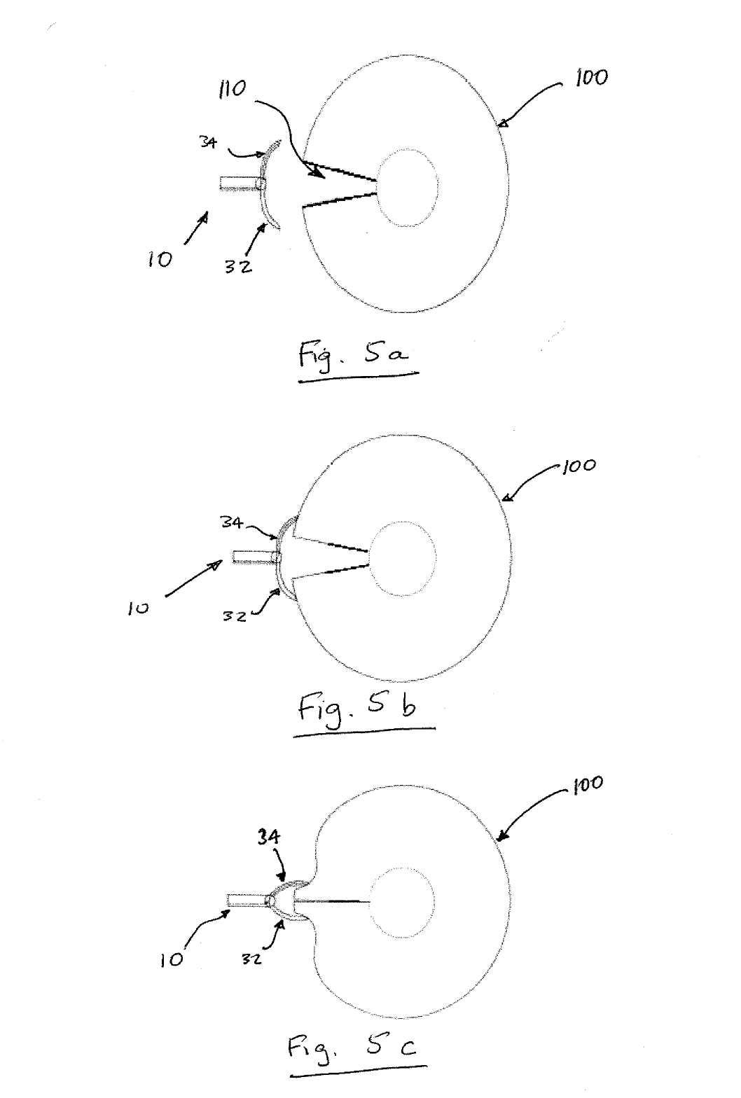

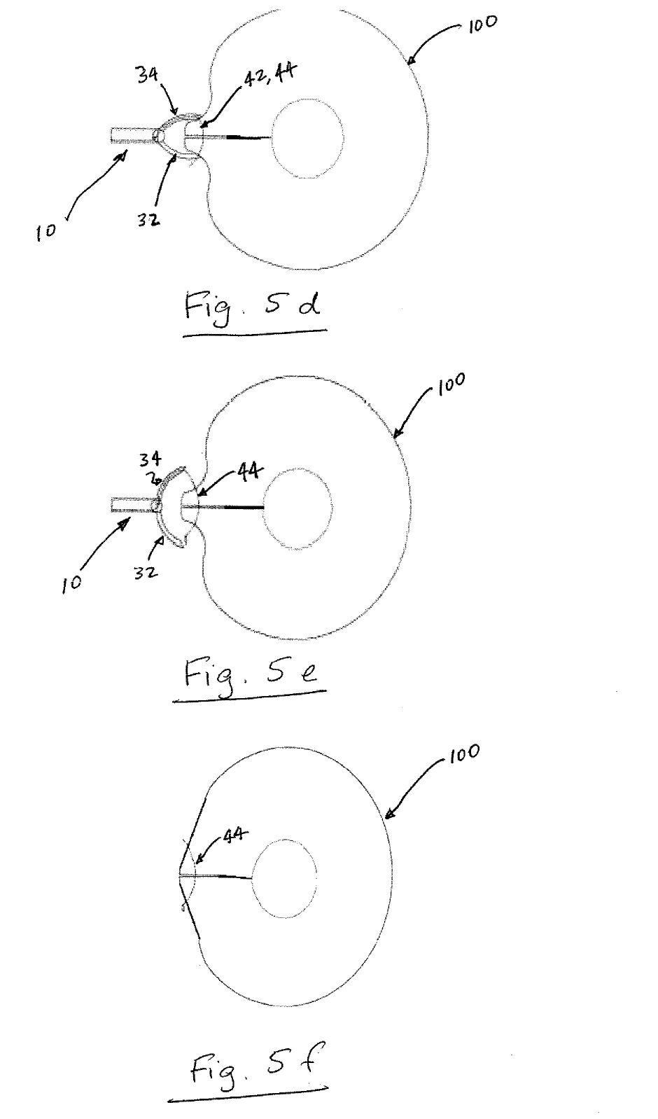

More particularly, FIG. 1 depicts device 10 comprising housing 12, distal end 14 and proximal end 16. Detail A of FIG. 1 provide a larger scale view of suturing assembly 30 comprising lower jaw 32 and upper jaw 34. Jaws 32 and 34 are used to grasp the tissue to be sutured and FIG. 1 show the jaws in the closed position. While jaws 32 and 34 are shown a being curved, the jaws may have other shapes, including less curved shapes and even straight shapes. The jaws may further comprise serrated tips or contain spikes or fangs which may be used at the jaw tips to help in initially grasping the tissue to be sutured. When suturing curvilinear tissue such as IVD or meniscus, curved-shaped jaws are most preferred as they are most adapted to grasping such tissue (see FIGS. 5a-f, e.g.). Jaws 32 and 34 are pivotally mounted with respect to each other by pivot 36 within housing 12. The ability of both of the jaws to be pivotable is a desirable feature in grasping tissue and the operator is not constrained by a fixed jaw design of other known suturing devices. Jaws 32 and 34 are linked by links 33 and 35, respectively to jaw actuator arm 40. Actuator arm 40 extends in the proximal direction through housing 12 to proximal end 16. Also depicted in Detail A is flexible needle 42 although some of the detail is obscured by the jaw assembly. Further discussion on the operation of needle 42 follows, particularly with reference to the discussion of FIG. 3 and FIG. 4.

Flexible needle 42 is made of a flexible material that offers short term memory capabilities including memory polymers (polypropylene, polyethylene, for example) or metallics (Nitinol, stainless steel sheets, Ti6Al4V, for example). One material exhibiting shape memory or super-elastic characteristics is Nitinol. Nitinol is utilized in a wide variety of applications, including medical device applications. Nitinol or NiTi alloys are widely utilized in the fabrication or construction of medical devices for a number of reasons, including its biomechanical compatibility, its biocompatibility, its fatigue resistance, its kink resistance, and its uniform plastic deformation. Other materials that have shape memory characteristics may also be used, for example, some polymers and metallic composition materials. It should be understood that these materials are not meant to limit the scope of the invention.

Detail B of FIG. 1, depicts further detail of proximal end 16 of device 10. In particular, proximal end 16 comprises handle 50, jaws actuator 52 and needle actuator 54. Jaws actuator 52 is linked directly to jaws actuator arm 40. Actuator 52 is pivotally connected to handle 50 by pivot 56 and controls the opening and closing of jaws 32 and 34. Needle actuator 54 is directly connected to flexible needle 42 and controls the extension and retraction of needle 42 from the jaws as explained in the discussion of FIGS. 3 and 4.

It should be noted that the particular description of proximal end 16 should not be considered limitative of the invention, just as a preferred embodiment. For example, what is called the jaws actuator 52 may become fixed and then function as the handle and what is the handle 50 may be made to pivot and modified to connect to the needle and thereby provide the leverage to function as the actuator of needle 42. Also, needle actuator 54, may be in the form of a trigger, designed into handle 50 or jaws actuator 52 rather than designed to be located on the side of housing 12 as shown in the FIG. 1.

FIG. 2 depicts an embodiment of device 10 with jaws 32 and 34 in the open position and needle 42 in the retracted position. Referring to details A and B of FIG. 2, jaws 32 and 34 have been actuated to the open position by movement of jaws actuator 52 (away from handle 50, in this embodiment) and corresponding movement of actuator arm 40 in a forward position. The relative degree of movement of actuator 52 may be observed by comparing the position of actuator 52 in Detail B of FIG. 1 with Detail B of FIG. 2.

FIG. 3 depicts an embodiment of device 10 with jaws 32 and 34 in the closed position, but with a cut away of jaw 32 which shows further detail of flexible needle 42. Specifically, referring to Detail A, needle 42 is shown to follow a channel within jaw 32. Needle 42 is a continuous throughout housing 12 and is jointed with needle actuator 54. Jaw 34 is also depicted to include opening 31 which is used to receive needle 42 as it is extended from jaw 32 (see FIG. 4 for more detail).

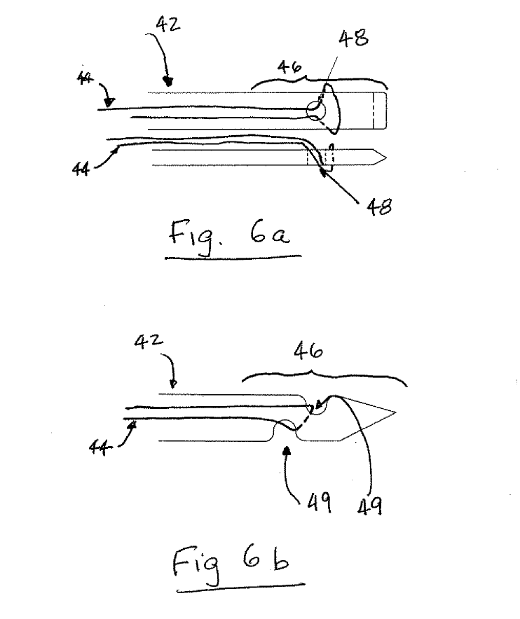

Finally, FIG. 4 depicts an embodiment of device 10 with jaws 32 and 34 in the closed position, but with needle 42 extending through opening 31. Extension of needle 42 from jaw 32 is achieved by advancing needle actuator 54 in the distal direction and correspondingly advancing needle 42 from its retracted position in jaw 32 to an extended position through opening 31 of jaw 34. Although not shown, suture 44 would be attached to the tip of needle 42 (see FIGS. 6 a-b for further needle tip detail). The relative movement of actuator 54 in advancing needle 42 can be observed by comparing the position of actuator 54 in Detail B of FIG. 3 with the position of actuator 54 as shown in Detail B of FIG. 4.

FIGS. 5 a-f are some idealized drawings of the operation of the device of this invention. FIG. 5a shows device 10 approaching intervertebral disc 100 having defect 110 after passage through a cannula or other minimally invasive site access tool (not shown). Jaws 32 and 34 are in an open configuration with flexible needle 42 retracted (not shown). FIG. 5b shows jaws 32 and 34 in an initial grasping position of disc 100. FIG. 5c shows jaws 32 and 34 after being actuated to the closed position. FIG. 5d shows actuation of flexible needle 42 to the extended position wherein flexible needle 42 has pierced disc 100. It should be noted that attached to needle 42 is suture 44. FIG. 5e shows jaws 32 and 34 actuated to the open position with needle 42 retracted and suture 44 in place. Prior to the opening of jaws 32 and 34 and retraction of needle 42, suture 44 is lifted out of the temporary holding place at the tip of needle 42 and grasped by small forceps or a similar tool prior to needle 42 being retracted and jaws 32 and 34 being opened. Finally, FIG. 5f depicts the sutured defect 110 of disc 100.

FIGS. 6 a-b show various flexible needle tip configurations for acceptance of suturing material 44. FIG. 6a shows a top view and a side view of tip 46 of needle 42 wherein hole 48 is shown for acceptance of suture 44. FIG. 6b shows an alternate tip configuration wherein suture 44 can be wrapped around indentations 49 for temporary securement prior to the suturing of tissue.

Thus in preferred embodiments, the device of this invention is used in methods where a suturing holder allows the insertion of suture 44 into the annulus fibrosus of intervertebral disc 100 or meniscus by using flexible needle 42 as a temporary guide for suture 44. Needle 42 is made out of a flexible metal alloy has indentation (s) 49, which serve(s) as a transient anchor point for suture 44. In use, flexible needle 42 is advanced through a channel or a guide in the needle and suture containing insertion jaw of the suturing assembly, which introduces the needle and suture into the annulus of disc 100 or other soft tissue. The insertion jaw of the suturing assembly has a unique curvature that bends needle 42 as it is passing through the tissue. The suturing assembly has also a receiving jaw that allows the capture of the suture 44. Once suture 44 has passed through the tissue, the suturing assembly is then retracted while suture 44 is kept in place in place by forceps or alternative tools which may access the suturing site through a secondary cannula.

Thus, the device of this invention is desirably used in a method of suturing soft tissue comprising the steps of:

i) providing tissue to be sutured;

ii) passing a needle carrying a suture through the tissue using a device having two independently pivotable tissue grasping jaws, wherein the first jaw houses the needle and provides a channel for the needle to have a retracted storage position and an extended tissue piercing position and the second jaw contains a channel for receiving the needle and suture after the needle and suture has passed through the tissue; and

iii) grasping and tying the suture after the suture has been passed through the tissue.

Preferred soft tissues to be sutured according to the method of this invention include those soft tissues that are curvilinear in shape such as IVD and meniscus.

Furthermore, with respect to how suture 44 is attached to needle 42, any conventional way known in the art may be employed. For example, simply passing suture 44 through hole 48 or around indentations 49 of needle tips as shown in FIGS. 6a and 6b are contemplated. More sophisticated methods of suturing may be employed such as may result with modification of jaws 32 and 34 to resemble the tips of the device as depicted and described in U.S. Pat. No. 3,842,840, the disclosure of which is incorporated by reference. Simply put, the device of this invention is contemplated to have jaws 32 and 34 modified to function in a manner that allows needle 42 and suture 44 to be stored in one of the jaws and as needle 42 is advanced, it picks up suture 44. After the needle and suture is driven through the tissue to be sutured, the needle and suture enters a needle-receiving passage of the second jaw which contains a spring-biased slide clamp which grips the suture as it initially passes with the needle and retains the suture as the needle is withdrawn.

Additionally, this invention contemplates that suture 44 may also simply be secured to needle 42 external to housing 12 of device 10. In this embodiment, needle 42 is extended beyond jaw 32 so as to allow one to secure suture 44 onto needle 42. Needle 44 with suture 42 is then retracted back in jaw 32. Alternatively, suture 44 may be secured onto needle 42 by first withdrawing needle 42 from the proximal end 16 of device 10, suture 44 is attached to needle 42 and then reinserted through proximal end 16 and through housing 12 to distal end 14.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A minimally invasive device for suture repair of soft tissue comprising:
a) an elongated housing having a proximal end and a distal end;
b) the distal end comprising a suturing assembly comprising:
(i) an upper jaw and a lower jaw wherein the upper and lower jaws are pivotally mounted with respect to each other and with respect to the housing;
(ii) a flexible needle adapted to carry a suture, the needle being movable between a first position and a second position and wherein the needle is substantially housed within and extendable from and retractable within one of the jaws and the housing and extends to the proximal end of the housing through the housing; and
c) the proximal end comprising a handle, a needle actuator and a jaws actuator, the needle actuator connected to the needle for extension and retraction of the needle from the needle-containing jaw, and the jaws actuator linked to the jaws within the housing for closing and opening of the jaws with respect to each other and for grasping and releasing the tissue to be sutured.

2. The device of claim 1, wherein the tips of the jaws further comprise serrations.

3. The device of claim 1, wherein the tips of the jaws further comprise spikes or fangs.

4. The device of claim 1, wherein the jaws are curve shaped.

5. The device of claim 4, wherein the tips of the jaws are serrated.

6. The device of claim 4, wherein the tips of the jaws further comprise spikes or fangs.

7. A method of suturing soft tissue comprising the steps of:
i) providing tissue to be sutured;
ii) passing a needle carrying a suture through the tissue using a device having two independently pivotable tissue grasping jaws, wherein the first jaw houses the needle and provides a channel for the needle to have a retracted storage position and an extended tissue piercing position and the second jaw contains a channel for receiving the needle and suture after the needle and suture has passed through the tissue; and
iii) grasping and tying the suture after the suture has been passed through the tissue.

8. The method of claim 7, wherein the tissue is the annulus fibrosus of an intervertebral disc.

9. The method of claim 7, wherein the tissue is meniscus.
